(19) United States
(12) Patent Application Publication
(10) Pub. No.: US 2021/0204594 A1
(43) Pub. Date: Jul. 8, 2021

(54) AEROSOL GENERATION

(71) Applicant: Nicoventures Trading Limited, London (GB)

(72) Inventors: Kelly Rees, London (GB); Richard Todd, London (GB)

(21) Appl. No.: 17/058,455

(22) PCT Filed: May 24, 2019
(86) PCT No.: PCT/EP2019/063501

§ 371 Date: Nov. 24, 2020

(30) Foreign Application Priority Data
May 24, 2018 (GB) 1808526.6

(51) Int. Cl. A24D 1/20; A24D 1/00; A24D 1/02; A24B 15/28; A24F 40/20; A24F 40/46; A24B 15/167

(52) U.S. Cl. CPC A24D 1/20; A24D 1/002; A24D 1/02; A24B 15/167; A24F 40/20; A24F 40/46; A24B 15/283

(57) ABSTRACT

The invention provides an aerosolizable material for use in an aerosol generating assembly, the aerosolizable material comprising a tobacco material, an un-encapsulated flavorant and an encapsulated flavorant.

### PRIORITY CLAIM

The present application is a National Phase entry of PCT Application No. PCT/EP2019/063501, filed May 24, 2019 which claims priority from GB Patent Application No. 1808526.6, filed May 24, 2018, each of which is hereby fully incorporated herein by reference.

### TECHNICAL FIELD

The present invention relates to aerosol generation and particularly, although not exclusively, to an aerosol generating assembly, a method of generating an aerosol, an aerosolizable material for use in generating an aerosol and an aerosol generating article for use in an aerosol generating assembly.

### BACKGROUND

Smoking articles such as cigarettes, cigars and the like burn tobacco during use to create tobacco smoke. Alternatives to these types of articles release compounds without burning.

Apparatus is known that heats aerosolizable material to volatilize at least one component of the aerosolizable material, typically to form an aerosol which can be inhaled, without burning or combusting the aerosolizable material. Such apparatus is sometimes described as a “heat-not-burn” apparatus or a “tobacco heating product” (THP) or “tobacco heating device” or similar. Various different arrangements for volatilizing at least one component of the aerosolizable material are known.

The material may be for example tobacco or other non-tobacco products or a combination, such as a blended mix, which may or may not contain nicotine.

Some known tobacco heating devices include more than one heater, with each heater configured to heat different parts of the aerosolizable material in use. This then allows the different parts of the aerosolizable material to be heated at different times and/or to different temperatures so as to provide longevity of aerosol formation over the use lifetime.

### SUMMARY

According to a first aspect of the present invention, there is provided an aerosolizable material for use in an aerosol generating assembly, the aerosolizable material comprising a tobacco material, an un-encapsulated flavorant and an encapsulated flavorant.

In some cases, the aerosolizable material is in the form of a component comprising at least two sections, and the two sections have different compositions.

In some cases, both sections comprise an un-encapsulated flavorant, and wherein only one of the two sections comprises an encapsulated flavorant.

In some cases, only one of the two sections comprises an un-encapsulated flavorant, and wherein only one of the two sections comprises an encapsulated flavorant. In some such cases, the un-encapsulated flavorant and the encapsulated flavorant are provided in different sections. In other such cases, the un-encapsulated flavorant and the encapsulated flavorant are provided in the same section.

In any of these cases, the tobacco material may be provided in either or both of the two sections. In some particular cases where the encapsulated flavorant is provided in only one section, tobacco material may be provided in at least the other section.

In some cases, the encapsulated flavorant is applied to a wrapper arranged around the tobacco material, suitably in the form of a film.

In some cases, the encapsulated flavorant provides a multi-modal flavorant release profile from the encapsulated flavorant on heating. In some cases, the encapsulated flavorant provides a bi-modal flavorant release profile from the encapsulated flavorant on heating.

In some cases, the aerosolizable material is in the form of a component that has a rod shape.

In some cases, the un-encapsulated flavorant comprises menthol and/or a cooling agent. In some cases, the encapsulated flavorant comprises menthol and/or a cooling agent.

In some cases, the encapsulated flavorant comprises an encapsulating material, and wherein the encapsulating material comprises at least one of a polysaccharide material; a cellulosic material; a gelatin; a gum; a protein material; a polyol matrix material; a gel; a wax; a polyurethane; polymerized, hydrolyzed ethylene vinyl acetate, a polyester, a polycarbonate, a polymethacrylate, a polyglycol, polyethylene, polystryrene, polypropylene, polyvinyl chloride or a mixture thereof or a mixture thereof.

A second aspect of the invention provides an aerosol generating article for use in an aerosol generating assembly, the article comprising an aerosolizable material according to the first aspect of the invention and a cooling element and/or a filter.

A third aspect of the invention provides an aerosol generating assembly comprising a heater and an aerosolizable material according to the first aspect, wherein the heater is arranged to heat the aerosolizable material in use to generate an aerosol.

In some cases, the aerosol generating assembly comprises a heater and an aerosolizable generating article according to the second aspect.

In some cases, the aerosolizable material comprises at least two sections, and wherein the assembly is configured to provide a different heat profile to each of the sections of aerosolizable material. In some cases, the sections have the same composition. In some cases, the sections have different compositions. In some cases, the assembly comprises at least two heaters which are arranged to respectively heat different sections of the aerosolizable material.

A further aspect of the invention provides a method of generating an aerosol comprising heating, in an aerosol generating assembly, an aerosolizable material, wherein the aerosolizable material comprises a tobacco material, an un-encapsulated flavorant and an encapsulated flavorant.

In some cases, the aerosol generating material comprises at least two sections, and wherein a different heat profile is provided to each section of the aerosolizable material. In some cases, the sections have different compositions.

### BRIEF DESCRIPTION OF THE DRAWINGS

Further features and advantages of the invention will become apparent from the following description of examples of the invention, given by way of example only, which is made with reference to the accompanying drawings.

FIG. 1 is a schematic view of an aerosolizable material for use in an aerosol generating assembly according to certain embodiments of the present invention.

FIG. 2 is a schematic view of an aerosol generating article comprising an aerosolizable material for use in an aerosol generating assembly according to certain embodiments of the present invention.

FIG. 3 is a sectional elevation view of an example of an aerosol generating article according to certain embodiments of the present invention.

FIG. 4 is a perspective view of the article of FIG. 3.

FIG. 5 is a sectional elevation view of an example of an aerosol generating article according to certain embodiments of the present invention.

FIG. 6 is a perspective view of the article of FIG. 5.

FIG. 7 is a perspective view of an example of an aerosol generating assembly according to certain embodiments of the present invention.

FIG. 8 is a sectional view of an example of an aerosol generating assembly according to certain embodiments of the present invention.

FIG. 9 is a perspective view of an example of an aerosol generating assembly according to certain embodiments of the present invention.

FIG. 10a shows a flavorant delivery profile for an example aerosol generating article according to certain embodiments of the present invention and two comparative flavorant delivery profiles.

FIG. 10b shows a heating profile that may be used in an aerosol generating assembly according to certain embodiments of the present invention.

FIG. 10c shows a flavorant delivery profile for two example aerosol generating articles according to certain embodiments of the present invention and one comparative flavorant delivery profile.

### DETAILED DESCRIPTION

Examples of the present invention provide an aerosolizable material for use in an aerosol generating assembly, the aerosolizable material comprising a tobacco material, an un encapsulated flavorant and an encapsulated flavorant.

The inventors have established that flavorants in tobacco heating products may be consumed rapidly at the beginning of the consumption experience due to their volatility. The present invention provides an aerosolizable material comprising (i) un-encapsulated flavorant, which is volatilized at the start of consumption period and (ii) encapsulated flavorant, which is released and volatilized later in the consumption period. This means that the claimed aerosolizable material may be used in a tobacco heating product and provide a sustained flavorant delivery (and a more sustained sensorial effect to the consumer) and, in some cases, a relatively constant flavorant delivery per puff (i.e. a relatively constant sensorial effect).

Encapsulation also serves to prevent migration of the flavorant within the aerosolizable material before use.

In some cases, the encapsulated flavorant is released once a threshold temperature, also referred to as the release temperature, is exceeded. In some cases, the temperature dependent release may be provided through use of an encapsulating material that that melts, decomposes, reacts, degrades, swells or deforms to release the flavorant at the release temperature. In other cases, heating may cause the encapsulated flavorant to swell causing rupture of the encapsulating material.

In some cases, the encapsulated flavorant may be present in the form of flavor capsules. In some cases, the encapsulated flavorant may be present in the form of powder, granules and/or beads. In some cases, the encapsulated flavorant may be present in the form of an encapsulating film which may be applied to, for example, the tobacco material and/or a wrapper arranged around the tobacco material. In some cases, the encapsulated flavorant may be present in a mixture of these forms, such as a combination of flavor capsules and an encapsulating film.

In some cases, the aerosolizable material may be configured for use in an aerosol generating assembly in which there is more than one heating zone. The aerosolizable material may be in the form of a component that comprises sections corresponding to each heating zone, wherein each section is subject to a different heat profile. In some cases, each section of the aerosolizable material may have substantially the same composition. In some other cases, each section of the aerosolizable material may have a different composition. For example, the aerosolizable material may comprise two sections and the un-encapsulated flavorant may be arranged in a different section to the encapsulated flavorant; a section of the aerosolizable material that is heated first in use may comprise the un-encapsulated t flavorant (but not encapsulated flavorant) and a section of the aerosolizable material that is heated second in use may comprise the encapsulated flavorant (but not un-encapsulated flavorant). In another example, both sections may comprise un-encapsulated flavorant but only the second may comprise an encapsulated flavorant. The inventors have determined that encapsulating the flavorant in later-heated sections limits consumption of the flavorant from those sections caused by heat bleeding from earlier-heated sections. In such cases, the encapsulated flavorant may be released when the release temperature is exceeded, which occurs only when the later-heated section is heated; heat bleeding from other sections is insufficient to exceed the release temperature. This arrangement then contributes to providing a sustained flavorant delivery profile.

In some cases both sections comprise an un-encapsulated flavorant, and wherein only one of the two sections comprises an encapsulated flavorant. In some other cases, only one of the two sections comprises an un-encapsulated flavorant, and wherein only one of the two sections comprises an encapsulated flavorant; the un-encapsulated flavorant and the encapsulated flavorant may be provided in the same section or in different sections. In any of these cases, a tobacco material may be provided in either or both of the two sections.

In some cases, the aerosolizable material may contain encapsulated flavorant, wherein the flavor is encapsulated to provide multi-modal release from the encapsulated flavorant on heating. That is, the flavorant release profile from the aerosolizable material includes a release from the un-encapsulated flavorant and at least two releases from the encapsulated flavorant. In some cases, the aerosolizable material may contain encapsulated flavorant, wherein the flavor is encapsulated to provide bi-modal release from the encapsulated flavorant on heating. That is, the flavorant release profile from the aerosolizable material includes a release from the un-encapsulated flavorant and two releases from the encapsulated flavorant. The flavorant release is staggered in use, providing sustained flavor delivery during the consumption experience.

Multi-modal (suitably bi-modal) flavorant release from encapsulated flavorant may be provided in a number of ways. In some cases, the release temperatures of the encapsulated flavorant may differ, such that release of the flavorant is staggered in use (providing the separate modes); the encapsulated flavorant with a lower release temperature is released and volatilized before the encapsulated flavorant with a higher release temperature. For example, the encapsulation material may differ in order to provide a multi-modal flavorant release profile; a first portion of the encapsulated flavorant employing an encapsulation material with a lower melting point may release the encapsulated flavorant before a second portion which is made using an encapsulation material with a high melting point. In another example, the encapsulated flavorant composition may differ; the encapsulated material may swell on heating in order to rupture the encapsulation, and different encapsulated flavorant compositions swell at different rates, thereby providing a multi-modal flavorant release profile. In another example, the encapsulated flavorant may have an at least similar release temperature throughout but the ratio of encapsulated material to encapsulating material may differ in order to provide a multi-modal flavorant release profile; encapsulated flavorant which includes a higher proportion of encapsulating material may require a longer period of heating in excess of the release temperature in order to release the flavorant.

In some cases, the encapsulated flavorant providing a multi-modal release profile may be arranged in the aerosol generating material in a non-uniform manner. For example, where the aerosol generating material has more than one section (which may correspond to different heating zones in use), the respective sections may contain different proportions of the encapsulated flavorant that correspond to each release mode. In some cases, the encapsulated flavorant providing the first release mode may be provided in a different section of the aerosolizable material to the encapsulated flavorant providing the second release mode.

In some cases, the un-encapsulated flavorant may comprise, consist essentially of or consist of menthol.

In some cases, the encapsulated flavorant may comprise, consist essentially of or consist of menthol.

The encapsulating material may be, for example, a polysaccharide or cellulosic material; a gelatin; a gum; a protein material; a polyol matrix material; a gel; a wax; a polyurethane; polymerized, hydrolyzed ethylene vinyl acetate, a polyester, a polycarbonate, a polymethacrylate, a polyglycol, polyethylene, polystryrene, polypropylene, polyvinyl chloride or a mixture thereof. Suitable polysaccharides include alginate, starch, dextran, maltodextrin, cyclodextrin and pectin. Suitable cellulosic materials include methyl cellulose, ethyl cellulose, hydroxyethyl cellulose, hydroxypropyl cellulose, carboxymethyl cellulose, and cellulose ethers. Suitable gums include gum Arabic, gum ghatti, gum tragacanth, Karaya, locust bean, acacia gum, guar, quince seed and xanthan gums. Suitable protein materials include zein proteins. Suitable polyol matrixes may be formed from polyvinyl alcohol. Suitable gels include agar, agarose, carrageenans, furoidan and furcellaran. Suitable waxes include camauba wax.

In some cases, the encapsulating material comprises a polysaccharide. In some particular cases, the encapsulating material may comprise an alginate. The alginate may be, for instance, a salt of alginic acid, an esterified alginate or glyceryl alginate. Salts of alginic acid include ammonium alginate, triethanolamine alginate, and group I or II metal ion alginates like sodium, potassium, calcium and magnesium alginate. Esterified alginates include propylene glycol alginate and glyceryl alginate.

In some cases, the encapsulating material is sodium alginate and/or calcium alginate. Calcium alginate provides a greater inhibition of migration of the flavorant at ambient temperature than sodium alginate, but also may release the aerosol generating agent at a higher temperature than the latter.

In some cases, the encapsulating material comprises a pectin.

In some cases, the aerosolizable material may additionally comprise one or more aerosol generating agents. In some cases, at least some of the aerosol generating agent may be encapsulated, optionally by the same material as the encapsulated flavorant.

In some cases, the aerosolizable material is in the form of a component that has a rod shape. It may additionally comprise a wrapper arranged around the tobacco material. One or more components of the aerosolizable material may be provided as a component of the wrapper. As used herein, the term “rod” generally refers to an elongate body which may be any suitable shape for use in an aerosol generating assembly. In some cases, the rod is substantially cylindrical.

In some cases, the aerosolizable material may be a solid material. In some cases, the aerosolizable material may comprise about 300-500 mg of tobacco material.

The encapsulated flavorant may be made according to any of several known methods widely disclosed in the art including, by way of example only, spray drying, fluidized bed coating, in-situ polymerization, solvent evaporation, coacervation, and/or coextrusion.

Examples of the invention also provide an aerosol generating assembly comprising a heater and an aerosolizable material according to the first aspect, wherein the heater is arranged to heat the aerosolizable material in use to generate an aerosol.

The aerosol generating assembly according to examples of the invention may also be referred to herein as a heat-not-burn device, a tobacco heating product or a tobacco heating device.

Any suitable heating profile may be employed. In some cases, the heater temperature may quickly rise at the beginning of a consumption session in order to generate an aerosol quickly. It may then fall after a period of time to prevent charring or combustion of the aerosolizable material. It may then rise again later in the session in order to maximize aerosolization of components of the material.

In some cases, the assembly is configured to provide a different heat profile to different sections of the aerosolizable material. In some cases, the assembly may be configured such that at least a portion of the aerosolizable material is exposed to a temperature of at least 180° C. or 200° C. for at least 50% of the heating period. In some examples, the aerosolizable material may be exposed to a heat profile as described in co-pending application PCT/EP2017/068804, the contents of which are incorporated herein in their entirety.

In some particular cases, an assembly is provided which is configured to heat the at least two sections of the aerosolizable material separately. By controlling the temperature of the first and second sections over time such that the temperature profiles of the sections are different, it is possible to control the puff profile of the aerosol during use. The heat provided to the two portions of the aerosolizable material may be provided at different times or rates; staggering the heating in this way may allow for both fast aerosol production and longevity of use.

In some cases, the assembly may be configured such that on initiation of the consumption experience, a first heating element corresponding to a first section of the aerosolizable material is immediately heated to a volatilization temperature which effects volatilization of the aerosolizable components. After a set period of time, the first heating element temperature drops to an intermediate temperature, which is selected to prevent condensation of the aerosol in the first section.

Either on initiation of the consumption experience or after period of time, a second heating element corresponding to a second section of the aerosolizable material is heated to an intermediate temperature (which may be the same or different to the first heating element intermediate temperature). After a set period of time, the second heating element is heated to a volatilization temperature (which may be the same or different to the first heating element volatilization temperature). Typically, at least one of the heating elements is at its volatilization temperature throughout the consumption experience, and in some cases, both heating elements are at their volatilization temperature simultaneously, for a short period of time. The second heating element intermediate temperature is selected so that the second section can be heated to its volatilization temperature quickly.

At the end of the consumption experience, both heating elements are allowed to cool to room temperature.

In one particular example, the assembly may be configured such that on initiation of the consumption experience, a first heating element corresponding to a first section of the aerosolizable material is immediately heated to a temperature of 240° C. This first heating element is maintained at 240° C. for 145 seconds and then drops to 135° C. (where it remains for the rest of the consumption experience). 75 seconds after initiation of the consumption experience, a second heating element corresponding to a second section of the aerosolizable material is heated to a temperature of 160° C. 135 seconds after initiation of the consumption experience, the temperature of the second heating element is raised to 240° C. (where it remains for the rest of the consumption experience). The consumption experience lasts 280 seconds, at which point both heaters are cool to room temperature.

In some cases, there are two sections in the aerosolizable material. In other cases, there may be 3, 4, 5, 6 or more sections. The composition of the aerosolizable material in each section may be the same or different. There may be un-encapsulated flavorant in any number of sections. There may be encapsulated flavorant in any number of sections. In some cases, the flavorant may be encapsulated so as to provide multi-modal release encapsulated flavorant on heating, and the aerosolizable material may be configured such that each mode is provided by a different section of the aerosolizable material. In some cases, the sections of aerosolizable material may comprise encapsulated flavorant which provides multi-modal release encapsulated flavorant on heating, wherein the proportion of encapsulated flavorant contributing to each release mode differs between the respective sections. In some cases, the assembly comprises a plurality of heaters, arranged such that each directly heats one or more sections of the aerosolizable material. In some cases, the number of heaters is equivalent to the number of sections in the aerosolizable material, and the heaters are arranged such that each heats one section.

In some cases, the aerosolizable material has a rod shape, such as a cylinder. In some cases, the sections of the aerosolizable material may be cylindrical and arranged coaxially along the rod of aerosolizable material. In other cases, the sections of the aerosolizable material may be in the form of prismatic sections that are arranged to together form a rod such as a cylinder. For example, in the case where there are two sections, they may be hemicylindrical and arranged with their respective planar faces in contact.

In some examples, the aerosolizable material may be provided as part of an aerosol generating article which is inserted into the aerosol generating assembly. In some cases, the aerosol generating article may comprise the aerosolizable material and additionally a cooling element and/or a filter. The cooling element, if present, may act or function to cool gaseous or aerosol components. In some cases, it may act to cool gaseous components such that they condense to form an aerosol. It may also act to space the very hot parts of the apparatus from the user. The filter, if present, may comprise any suitable filter known in the art such as a cellulose acetate plug. In some cases, the filter does not include or contain any encapsulated flavorant. The aerosol generating article may be circumscribed by a wrapping material such as paper.

The aerosol generating article may additionally comprise ventilation apertures. These may be provided in the sidewall of the article. In some cases, the ventilation apertures may be provided in the filter and/or cooling element. These apertures may allow cool air to be drawn into the article during use, which can mix with the heated volatilized components thereby cooling the aerosol.

The ventilation enhances the generation of visible heated volatilized components from the article when it is heated in use. The heated volatilized components are made visible by the process of cooling the heated volatilized components such that supersaturation of the heated volatilized components occurs. The heated volatilized components then undergo droplet formation, otherwise known as nucleation, and eventually the size of the aerosol particles of the heated volatilized components increases by further condensation of the heated volatilized components and by coagulation of newly formed droplets from the heated volatilized components.

In some cases, the ratio of the cool air to the sum of the heated volatilized components and the cool air, known as the ventilation ratio, is at least 15%. A ventilation ratio of 15% enables the heated volatilized components to be made visible by the method described above. The visibility of the heated volatilized components enables the user to identify that the volatilized components have been generated and adds to the sensory experience of the smoking experience.

In another example, the ventilation ratio is between 50% and 85% to provide additional cooling to the heated volatilized components. In some cases, the ventilation ratio may be at least 60% or 65%.

As used herein, an “aerosol generating agent” is an agent that promotes the generation of an aerosol on heating. An aerosol generating agent may promote the generation of an aerosol by promoting an initial vaporization and/or the condensation of a gas to an inhalable solid and/or liquid aerosol. Suitable aerosol generating agents include, but are not limited to: a polyol such as sorbitol, glycerol, and glycols like propylene glycol or triethylene glycol; a non-polyol such as monohydric alcohols, high boiling point hydrocarbons, acids such as lactic acid, glycerol derivatives, esters such as diacetin, triacetin, triethylene glycol diacetate, triethyl citrate or myristates including ethyl myristate and isopropyl myristate and aliphatic carboxylic acid esters such as methyl stearate, dimethyl dodecanedioate and dimethyl tetradecanedioate.

As used herein, the terms “flavor” and “flavorant” refer to materials which, where local regulations permit, may be used to create a desired taste or aroma in a product for adult consumers. They may include extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder. In some embodiments, the sensonal receptor site activator or stimulator is a sensate, such as a cooling agent. Suitable cooling agents may comprise one or more compounds selected from the group consisting of: N-ethyl-2-isopropyl-5-methylcyclo hexane carboxamide (also known as WS-3, CAS: 39711-79-0, FEMA: 3455); 2-isopropyl-N-[(ethoxycarbonyl)methyl]-5-methylcyclohexanecarboxamide (also known as WS-5, CAS: 68489-14-5, FEMA: 4309); 2-isopropyl-N-(4-methoxylphenyl)-5-methylcyclohexanecarboxamide (also known as WS-12, FEMA: 4681); and 2-isopropyl-N,2,3-trimethylbutanamide (also known as WS-23, FEMA: 3804).

As used herein, the term “tobacco material” refers to any material comprising tobacco or derivatives therefore. The term “tobacco material” may include one or more of tobacco, tobacco derivatives, expanded tobacco, reconstituted tobacco or tobacco substitutes. The tobacco material may comprise one or more of ground tobacco, tobacco fiber, cut tobacco, extruded tobacco, tobacco stem, reconstituted tobacco and/or tobacco extract.

The tobacco used to produce tobacco material may be any suitable tobacco, such as single grades or blends, cut rag or whole leaf, including Virginia and/or Burley and/or Oriental. It may also be tobacco particle ‘fines’ or dust, expanded tobacco, stems, expanded stems, and other processed stem materials, such as cut rolled stems. The tobacco material may be a ground tobacco or a reconstituted tobacco material. The reconstituted tobacco material may comprise tobacco fibers, and may be formed by casting, a Fourdrinier-based paper making-type approach with back addition of tobacco extract, or by extrusion.

In use, in some cases, the aerosol generating article may be arranged in an aerosol generating device which heats the article to generate an aerosol without burning. In some other cases, the article may be provided in an assembly with a fuel source, such as a combustible fuel source or chemical heat source, which heats but does not burn the aerosolizable material.

In some cases, the heater provided in an aerosol generating assembly may be a thin film, electrically resistive heater. In other cases, the heater may comprise an induction heater or the like. Where more than one heater is present, each heater may be the same or different.

Generally, the or each heater is connected to a battery, which may be a rechargeable battery or a non-rechargeable battery. Examples of suitable batteries include for example a lithium-ion battery, a nickel battery (such as a nickel-cadmium battery), an alkaline battery and/or the like. The battery is electrically coupled to the heater and is controllable via appropriate circuitry to supply electrical power when required to heat the aerosolizable material (to volatilize components of the aerosolizable material without causing the aerosolizable material to burn).

In one example, the heater is generally in the form of a hollow cylindrical tube, having a hollow interior heating chamber into which the aerosolizable material is inserted for heating in use. Different arrangements for the heater are possible. For example, the heater may be formed as a single heater or may be formed of plural heaters aligned along the longitudinal axis of the heater. (For simplicity, reference to a “heater” herein shall be taken to include plural heaters, unless the context requires otherwise.) The heater may be annular or tubular. The heater may be dimensioned so that substantially the whole of the aerosolizable material when inserted is located within the heating element(s) of the heater so that substantially the whole of the aerosolizable material is heated in use. The heater may be arranged so that selected zones of the aerosolizable material can be independently heated, for example in turn (sequentially) or together (simultaneously) as desired.

The heater may be surrounded along at least part of its length by a thermal insulator which helps to reduce heat passing from the heater to the exterior of the aerosol generating assembly. This helps to keep down the power requirements for the heater as it reduces heat losses generally. The insulator also helps to keep the exterior of the aerosol generating assembly cool during operation of the heater.

To the extent that they are compatible, features described in relation to one aspect of the invention are explicitly disclosed in combination with the other aspects and examples described herein.

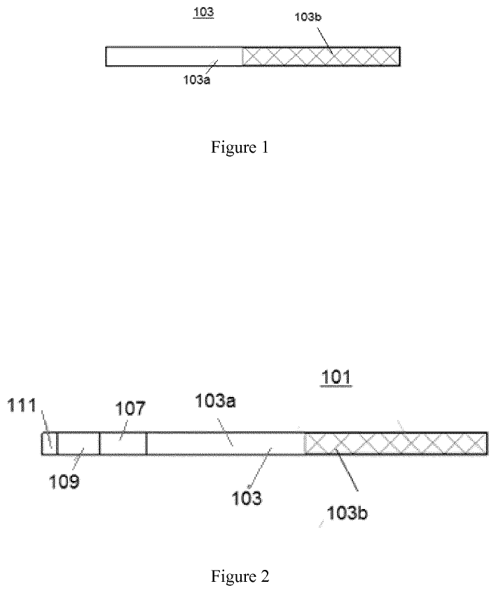

FIG. 1 illustrates schematically an example of an aerosolizable material for use with an aerosol generating assembly. The aerosolizable material is in the form of a cylindrical rod and comprises a first section 103a and a second section 103b. The second section 103b is, in this example, further from the mouth in use than the first section 103a.

In some examples, the two sections 103a, 103b of aerosolizable material have substantially the same composition. They comprise a tobacco material, un-encapsulated flavorant and encapsulated flavorant. The flavorant may be menthol. The encapsulating material may be an alginate. In some cases, the encapsulated flavorant may be in the form of capsules which are dispersed through the tobacco material. In some other cases, the encapsulated flavorant may be provided in the form of a film that is applied to a wrapper (such as a paper wrapper) arranged around the tobacco material. In such cases, the film may be applied by spray drying or printing, for example. In yet further cases, the encapsulated flavorant may be applied to the tobacco material, for example by spraying.

In other examples, the two sections 103a, 103b of aerosolizable material have substantially the same composition. They comprise a tobacco material, un-encapsulated flavorant and an encapsulated flavorant that provides a multi-modal release of the flavorant from the encapsulated flavorant on heating. In use, the un-encapsulated flavorant is volatilized initially, followed by release and volatilization of a first portion of the encapsulated flavorant (which may, in some cases, be the portion with the lower release temperature). The second portion of encapsulated flavorant (which may in some cases, be the portion with the higher release temperature) is released later and then volatilized providing staggered release of the flavorant and a sustained flavorant delivery to the user.

In other examples, the two sections 103a, 103b of aerosolizable material have different compositions. In some cases, both sections comprise un-encapsulated flavorant, but only one section comprises encapsulated flavorant. In other cases, one section comprises un-encapsulated flavorant but no encapsulated flavorant and the other comprises encapsulated flavorant but no un-encapsulated flavorant. In each case, either or both sections 103a, 103b may comprise a tobacco material. In yet other cases, one section comprises un-encapsulated flavorant and encapsulated flavorant (and optionally tobacco material) and the other section includes a tobacco material but does not include any flavorant. In some cases, the encapsulated flavorant may be in the form of capsules which are dispersed through the tobacco material in one section of the aerosolizable material. In some other cases, a wrapper (such as a paper wrapper) may be arranged around the tobacco material and the encapsulated flavorant may be provided in the form of a film that is applied to a section of the wrapper that is arranged around one of the sections of aerosolizable material 103a, 103b. In such cases, the film may be applied by spray drying or printing, for example. In yet further cases, the encapsulated flavorant may be applied to the tobacco material in one of the sections 103a, 103b, for example by spraying.

In such examples, the section of aerosolizable material that comprises the encapsulated flavorant will generally be the section that is configured to be heated later in use. In some cases, only the second section 103b (which is further from the mouth end) contains encapsulated flavorant and is heated after the first section 103 a, in use.

In other examples, the two sections 103a, 103b of aerosolizable material have different compositions. In some cases, either or both sections comprise un-encapsulated flavorant, and each section comprises encapsulated flavorant. The encapsulated flavorant is released from the encapsulated flavorant on heating in a bi-modal release profile; the encapsulated flavorant providing the first release mode is provided in a different section of the aerosolizable material to the encapsulated flavorant providing the second release mode. In each case, either or both sections 103a, 103b may comprise a tobacco material. In general, the section containing the encapsulated flavorant with a higher release temperature (or otherwise configured to provide later release) is the section that is heated later in use. For example, in one embodiment, the first section 103a comprises un-encapsulated flavorant and a first encapsulated flavorant. In this embodiment, the second section 103b comprises a second encapsulated flavorant which has a higher release temperature (or is otherwise configured to provide later release) than the first encapsulated flavorant. In use, the first section 103a is heated first and the un-encapsulated flavorant is volatilized initially, followed by the encapsulated flavorant from the first section when the release temperature is reached. The second encapsulated flavorant is volatilized at this stage as it has a higher release temperature (or is otherwise configured to provide later release); on heating of the second section 103b, the second encapsulated flavorant is released and volatilized to form an aerosol.

In a yet further variant, the two sections 103a, 103b of aerosolizable material have different compositions. In some cases, either of both sections comprise unencapsulated flavorant, and each section comprises encapsulated flavorant. The encapsulated flavorant is released from the encapsulated flavorant on heating in a bi-modal release profile; the respective sections of aerosol generating material comprise different proportions of the encapsulated flavorant that contribute to each of the release modes. For example, a first section of aerosolizable material comprises a greater proportion of the encapsulated flavorant providing the first release mode and a second section of aerosolizable material comprises a greater proportion of the encapsulated flavorant providing the second release mode. In each case, either or both sections 103a, 103b may comprise a tobacco material. Generally, the section containing a greater proportion of the encapsulated flavorant providing the second release mode will be heated second in use. In some cases, the second section may be the section 103b arranged further from the mouth in use.

FIG. 2 illustrates schematically an example of an aerosol generating article 101 for use with an aerosol generating assembly. The aerosol generating article 101 includes, the cylindrical rod of aerosolizable material 103 illustrated in FIG. 1, a cooling element 107, a filter 109 and a mouth-end segment 111. The cooling element 107 and filter 109, as illustrated, may be arranged between the mouth-end of the aerosolizable material 103 and the mouth-end segment 111, so that flow from the aerosolizable material 103 passes through the cooling element 107 and filter 109 (or vice versa if the filter is arranged before the cooling element in the flow) before reaching the user. Although the example in FIG. 2 illustrates a cooling element 107, a filter 109 and a mouth-end segment 111, one or more of these elements may be omitted in other examples.

In some examples, the mouth-end segment, if present, 111 may be formed of for example paper, for example in the form of a spirally wound paper tube, cellulose acetate, cardboard, crimped paper, such as crimped heat resistant paper or crimped parchment paper, and/or polymeric materials, such as low density polyethylene (LDPE), or some other suitable material. The mouth-end segment 111 may comprise a hollow tube. Such a hollow tube may provide a filtering function to filter volatilized aerosolizable material. The mouth-end segment 111 may be elongate, in order to be spaced from the very hot part(s) of the main apparatus (not shown) that heats the aerosolizable material.

In some examples, the filter 109, if present, may be a filter plug, and may be made, for example, from cellulose acetate.

In some cases, the cooling element 107, if present, may comprise a monolithic rod having first and second ends and comprising plural through holes extending between the first and second ends. The through holes may extend substantially parallel to the central longitudinal axis of the rod. The through holes of the cooling element 107 may be arranged generally radially of the element when viewed in lateral cross-section. That is, in an example, the element has internal walls which define the through holes and which have two main configurations, namely radial walls and central walls. The radial walls extend along radii of the cross-section of the element and the central walls are centered on the center of the cross-section of the element. The central walls in one example are circular, though other regular or irregular cross-sectional shapes may be used. Likewise, the cross-section of the element in one example is circular, though other regular or irregular cross-sectional shapes may be used.

In an example, the majority of the through holes have a hexagonal or generally hexagonal cross-sectional shape. In this example, the element has what might be termed a “honeycomb” structure when viewed from one end.

In some cases, the cooling element 107 may comprise a hollow tube which spaces the filter 109, if present, from the very hot part(s) of the main apparatus that heats the aerosolizable material. The cooling element 107 may be formed of for example paper, for example in the form of a spirally wound paper tube, cellulose acetate, cardboard, crimped paper, such as crimped heat resistant paper or crimped parchment paper, and polymeric materials, such as low density polyethylene (LDPE), or some other suitable material.

The cooling element 107, if present, may be substantially incompressible. It may be formed of a ceramic material, or of a polymer, for example a thermoplastic polymer, which may be an extrudable plastics material. In an example, the porosity of the element is in the range 60% to 75%. The porosity in this sense may be a measure of the percentage of the lateral cross-sectional area of the element occupied by the through holes. In an example, the porosity of the element is around 69% to 70%.

Other examples of a cooling element are disclosed in PCT/GB2015/051253, the entirety of which is hereby expressly incorporated by reference, in particular in FIGS. 1 to 8 and the description from page 8, line 11 to page 18, line 16.

In further examples, the cooling element 107 may be formed from a sheet material that is folded, crimped or pleated to form through holes. The sheet material may be made, for example, from metal such as aluminum; polymeric plastics material such as polyethylene, polypropylene, polyethylene terephthalate, or polyvinyl chloride; or paper.

In some examples, the cooling element 107 and the filter 109 may be held together by a wrapper paper (not shown) to form an assembly. The assembly may then be joined to the aerosolizable material by a further wrapper (not shown) which circumscribes the assembly and at least the mouth end of the aerosolizable material to form the aerosol generating article 101. In other examples, the aerosol generating article 101 is formed by wrapping the cooling element 107, the filter 109 and the aerosolizable material 103 effectively in one operation, with no separate tipping paper being provided for the cooling element and/or filter components (if present).

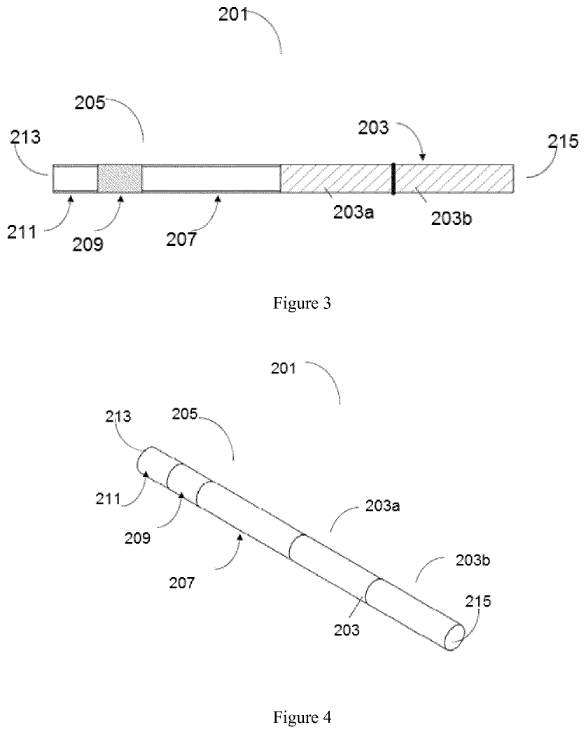

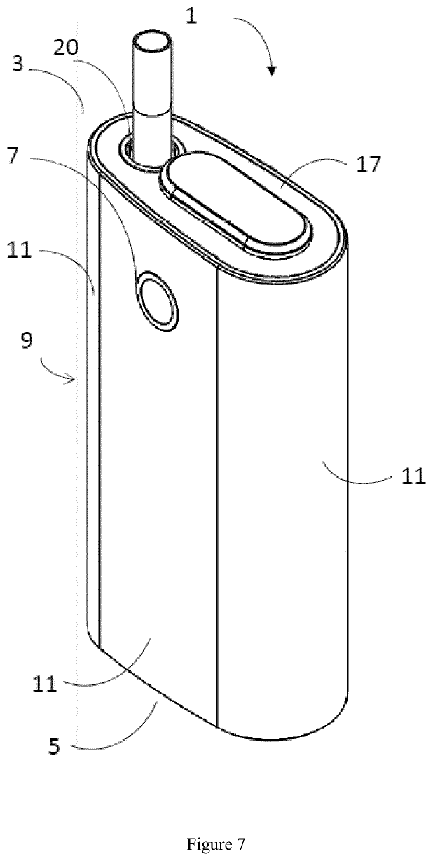

Referring now to FIGS. 3 and 4, there are shown a partially cut-away section view and a perspective view of an example of an aerosol generating article 201. The article 201 is adapted for use with device having a power source and a heater. The article 201 of this embodiment is particularly suitable for use with the device 1 shown in FIGS. 7 to 9, described below. In use, the article 201 may be removably inserted into the device shown in FIG. 7 at an insertion point 20 of the device 1. The reference signs shown in FIGS. 3 and 4 are equivalent to the reference signs shown in FIGS. 1 and 2, but with an increment of 100.

The article 201 of one example is in the form of a substantially cylindrical rod that includes an aerosolizable material 203 and a filter assembly 205 in the form of a rod. The aerosolizable material has two sections 203a, 203b; the above description of sections 103a, 103b from FIGS. 1 and 2 applies also to the sections 203a, 203b from FIGS. 3 and 4.

The filter assembly 205 includes three segments, a cooling segment 207, a filter segment 209 and a mouth end segment 211. The article 201 has a first end 213, also known as a mouth end or a proximal end and a second end 215, also known as a distal end. The aerosolizable material 203 is located towards the distal end 215 of the article 201. In one example, the cooling segment 207 is located adjacent to the aerosolizable material 203 between the aerosolizable material 203 and the filter segment 209, such that the cooling segment 207 is in an abutting relationship with the aerosolizable material 203 and the filter segment 209. In other examples, there may be a separation between the aerosolizable material 203 and the cooling segment 207 and between the aerosolizable material 203 and the filter segment 209. The filter segment 209 is located in between the cooling segment 207 and the mouth end segment 211. The mouth end segment 211 is located towards the proximal end 213 of the article 201, adjacent to the filter segment 209. In one example, the filter segment 209 is in an abutting relationship with the mouth end segment 211. In one embodiment, the total length of the filter assembly 205 is between 37 mm and 45 mm, suitably 41 mm.

In some examples, the aerosolizable material 203 is between 30 mm and 54 mm in length, suitably between 36 mm and 48 mm in length. In one example, the total length of the article 201 is between 71 mm and 95 mm, suitably between 79 mm and 87 mm, suitably about 83 mm.

An axial end of the aerosolizable material 203 is visible at the distal end 215 of the article 201. However, in other embodiments, the distal end 215 of the article 201 may comprise an end member (not shown) covering the axial end of the aerosolizable material 203.

The aerosolizable material 203 is joined to the filter assembly 205 by annular tipping paper (not shown), which is located substantially around the circumference of the filter assembly 205 to surround the filter assembly 205 and extends at least partially along the length of the aerosolizable material 203. In one example, the tipping paper is made of 58GSM standard tipping base paper. In one example, the tipping paper has a length of between 42 mm and 50 mm, suitably about 46 mm.

In some cases, the same tipping paper may be used to join the sections 203a, 203b of aerosolizable material 203 and the filter assembly 205.

In one example, the cooling segment 207 is an annular tube and is located around and defines an air gap within the cooling segment. The air gap provides a chamber for heated volatilized components generated from the aerosolizable material 203 to flow. The cooling segment 207 is hollow to provide a chamber for aerosol accumulation yet rigid enough to withstand axial compressive forces and bending moments that might arise during manufacture and whilst the article 201 is in use during insertion into the device 1. In one example, the thickness of the wall of the cooling segment 207 is approximately 0.29 mm.

The cooling segment 207 provides a physical displacement between the aerosolizable material 203 and the filter segment 209. The physical displacement provided by the cooling segment 207 will provide a thermal gradient across the length of the cooling segment 207. In one example the cooling segment 207 is configured to provide a temperature differential of at least 40 degrees Celsius between a heated volatilized component entering a first end of the cooling segment 207 and a heated volatilized component exiting a second end of the cooling segment 207. In one example the cooling segment 207 is configured to provide a temperature differential of at least 60 degrees Celsius between a heated volatilized component entering a first end of the cooling segment 207 and a heated volatilized component exiting a second end of the cooling segment 207. This temperature differential across the length of the cooling element 207 protects the temperature sensitive filter segment 209 from the high temperatures of the aerosolizable material 203 when it is heated by the heating arrangement of the device 1. If the physical displacement was not provided between the filter segment 209 and the aerosolizable material 203 and the heating elements of the device 1, then the temperature sensitive filter segment may 209 become damaged in use, so it would not perform its required functions as effectively.

In one example the length of the cooling segment 207 is at least 15 mm. In one example, the length of the cooling segment 207 is between 20 mm and 30 mm, suitably 23 mm to 27 mm or 25 mm to 27 mm, most suitably about 25 mm.

The cooling segment 207 may be made of paper, which means that it comprises a material that does not generate compounds of concern, for example, toxic compounds when in use adjacent to the heater arrangement of the device 1. In one example, the cooling segment 207 is manufactured from a spirally wound paper tube which provides a hollow internal chamber yet maintains mechanical rigidity. Spirally wound paper tubes are able to meet the tight dimensional accuracy requirements of high-speed manufacturing processes with respect to tube length, outer diameter, roundness and straightness.

In another example, the cooling segment 207 is a recess created from stiff plug wrap or tipping paper. The stiff plug wrap or tipping paper is manufactured to have a rigidity that is sufficient to withstand the axial compressive forces and bending moments that might arise during manufacture and whilst the article 201 is in use during insertion into the device 1.

The filter segment 209 may be formed of any filter material sufficient to remove one or more volatilized compounds from heated volatilized components from the aerosolizable material. In one example the filter segment 209 is made of a mono-acetate material, such as cellulose acetate. The filter segment 209 provides cooling and irritation-reduction from the heated volatilized components without depleting the quantity of the heated volatilized components to an unsatisfactory level for a user.

The density of the cellulose acetate tow material of the filter segment 209 controls the pressure drop across the filter segment 209, which in turn controls the draw resistance of the article 1. Therefore the selection of the material of the filter segment 209 is important in controlling the resistance to draw of the article 201. In addition, the filter segment performs a filtration function in the article 201.

In one example, the filter segment 209 is made of an 8Y15 grade of filter tow material, which provides a filtration effect on the heated volatilized material, whilst also reducing the size of condensed aerosol droplets which result from the heated volatilized material which consequentially reduces the irritation and throat impact of the heated volatilized material to satisfactory levels.

The presence of the filter segment 209 provides an insulating effect by providing further cooling to the heated volatilized components that exit the cooling segment 207. This further cooling effect reduces the contact temperature of the user's lips on the surface of the filter segment 209.

One or more flavors may be added to the filter segment 209 in the form of either direct injection of flavored liquids into the filter segment 209 or by embedding or arranging one or more flavored breakable capsules or other flavor carriers within the cellulose acetate tow of the filter segment 209.

In one example, the filter segment 209 is between 6 mm to 10 mm in length, suitably about 8 mm.

The mouth end segment 211 is an annular tube and is located around and defines an air gap within the mouth end segment 211. The air gap provides a chamber for heated volatilized components that flow from the filter segment 209. The mouth end segment 211 is hollow to provide a chamber for aerosol accumulation yet rigid enough to withstand axial compressive forces and bending moments that might arise during manufacture and whilst the article is in use during insertion into the device 1. In one example, the thickness of the wall of the mouth end segment 211 is approximately 0.29 mm.

In one example, the length of the mouth end segment 211 is between 6 mm to 10 mm and suitably about 8 mm.

The mouth end segment 211 may be manufactured from a spirally wound paper tube which provides a hollow internal chamber yet maintains critical mechanical rigidity. Spirally wound paper tubes are able to meet the tight dimensional accuracy requirements of high-speed manufacturing processes with respect to tube length, outer diameter, roundness and straightness.

The mouth end segment 211 provides the function of preventing any liquid condensate that accumulates at the exit of the filter segment 209 from coming into direct contact with a user. It should be appreciated that, in one example, the mouth end segment 211 and the cooling segment 207 may be formed of a single tube and the filter segment 209 is located within that tube separating the mouth end segment 211 and the cooling segment 207.

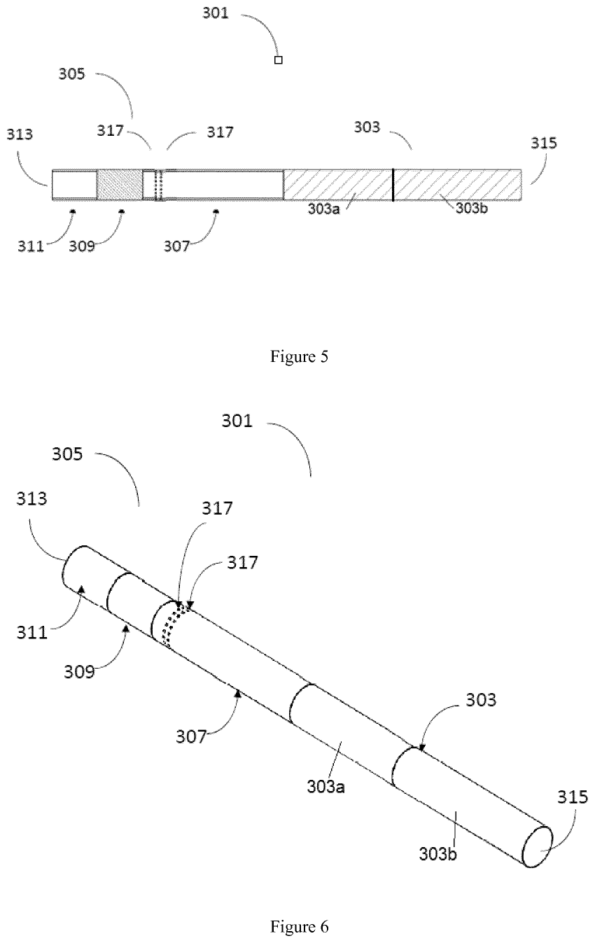

Referring now to FIGS. 5 and 6, there are shown a partially cut-away section and perspective views of an example of an article 301 according to an embodiment of the invention. The reference signs shown in FIGS. 5 and 6 are equivalent to the reference signs shown in FIGS. 3 and 4, but with an increment of 100.

In the example of the article 301 shown in FIGS. 5 and 6, a ventilation region 317 is provided in the article 301 to enable air to flow into the interior of the article 301 from the exterior of the article 301. In one example the ventilation region 317 takes the form of one or more ventilation holes 317 formed through the outer layer of the article 301. The ventilation holes may be located in the cooling segment 307 to aid with the cooling of the article 301. In one example, the ventilation region 317 comprises one or more rows of holes, and in some case, each row of holes is arranged circumferentially around the article 301 in a cross-section that is substantially perpendicular to a longitudinal axis of the article 301.

In one example, there are between one to four rows of ventilation holes to provide ventilation for the article 301. Each row of ventilation holes may have between 12 to 36 ventilation holes 317. The ventilation holes 317 may, for example, be between 100 to 500 mhi in diameter. In one example, an axial separation between rows of ventilation holes 317 is between 0.25 mm and 0.75 mm, suitably 0.5 mm.

In one example, the ventilation holes 317 are of uniform size. In another example, the ventilation holes 317 vary in size. The ventilation holes can be made using any suitable technique, for example, one or more of the following techniques: laser technology, mechanical perforation of the cooling segment 307 or pre-perforation of the cooling segment 307 before it is formed into the article 301. The ventilation holes 317 are positioned so as to provide effective cooling to the article 301.

In one example, the rows of ventilation holes 317 are located at least 11 mm from the proximal end 313 of the article, suitably between 17 mm and 20 mm from the proximal end 313 of the article 301. The location of the ventilation holes 317 is positioned such that user does not block the ventilation holes 317 when the article 301 is in use.

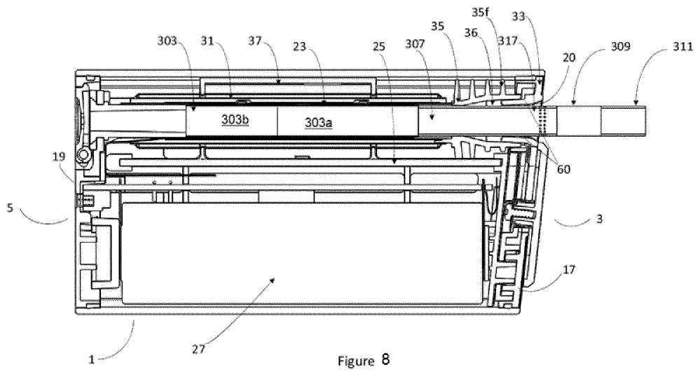

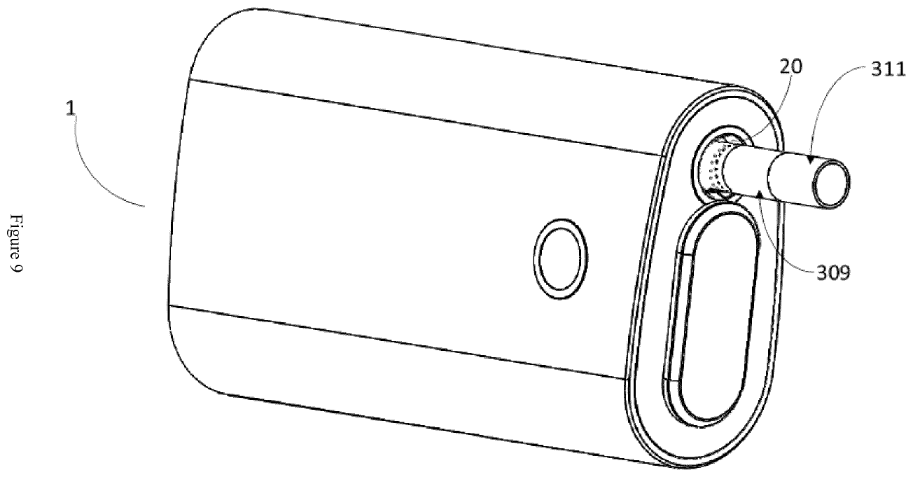

Providing the rows of ventilation holes between 17 mm and 20 mm from the proximal end 313 of the article 301 enables the ventilation holes 317 to be located outside of the device 1, when the article 301 is fully inserted in the device 1, as can be seen in FIGS. 8 and 9. By locating the ventilation holes outside of the device, non-heated air is able to enter the article 301 through the ventilation holes from outside the device 1 to aid with the cooling of the article 301.

The length of the cooling segment 307 is such that the cooling segment 307 will be partially inserted into the device 1, when the article 301 is fully inserted into the device 1. The length of the cooling segment 307 provides a first function of providing a physical gap between the heater arrangement of the device 1 and the heat sensitive filter arrangement 309, and a second function of enabling the ventilation holes 317 to be located in the cooling segment, whilst also being located outside of the device 1, when the article 301 is fully inserted into the device 1. As can be seen from FIGS. 8 and 9, the majority of the cooling element 307 is located within the device 1. However, there is a portion of the cooling element 307 that extends out of the device 1. It is in this portion of the cooling element 307 that extends out of the device 1 in which the ventilation holes 317 are located.

Referring now to FIGS. 7 to 9 in more detail, there is shown an example of a device 1 arranged to heat aerosolizable material to volatilize at least one component of the said aerosolizable material, typically to form an aerosol which can be inhaled. The device 1 is a heating device 1 which releases compounds by heating, but not burning, the aerosolizable material.

A first end 3 is sometimes referred to herein as the mouth or proximal end 3 of the device 1 and a second end 5 is sometimes referred to herein as the distal end 5 of the device 1. The device 1 has an on/off button 7 to allow the device 1 as a whole to be switched on and off as desired by a user.

The device 1 comprises a housing 9 for locating and protecting various internal components of the device 1. In the example shown, the housing 9 comprises a uni-body sleeve 11 that encompasses the perimeter of the device 1, capped with a top panel 17 which defines generally the ‘top’ of the device 1 and a bottom panel 19 which defines generally the ‘bottom’ of the device 1. In another example the housing comprises a front panel, a rear panel and a pair of opposite side panels in addition to the top panel 17 and the bottom panel 19.

The top panel 17 and/or the bottom panel 19 may be removably fixed to the uni-body sleeve 11, to permit easy access to the interior of the device 1, or may be “permanently” fixed to the uni-body sleeve 11, for example to deter a user from accessing the interior of the device 1. In an example, the panels 17 and 19 are made of a plastics material, including for example glass-filled nylon formed by injection molding, and the uni-body sleeve 11 is made of aluminum, though other materials and other manufacturing processes may be used.

The top panel 17 of the device 1 has an opening 20 at the mouth end 3 of the device 1 through which, in use, the article 201, 301 including aerosolizable material may be inserted into the device 1 and removed from the device 1 by a user.

The housing 9 has located or fixed therein a heater arrangement 23, control circuitry 25 and a power source 27. In this example, the heater arrangement 23, the control circuitry 25 and the power source 27 are laterally adjacent (that is, adjacent when viewed from an end), with the control circuitry 25 being located generally between the heater arrangement 23 and the power source 27, though other locations are possible.

The control circuitry 25 may include a controller, such as a microprocessor arrangement, configured and arranged to control the heating of the aerosolizable material in the consumable article 201, 301 as discussed further below.

The power source 27 may be for example a battery, which may be a rechargeable battery or a non-rechargeable battery. Examples of suitable batteries include for example a lithium-ion battery, a nickel battery (such as a nickel-cadmium battery), an alkaline battery and/or the like. The battery 27 is electrically coupled to the heater arrangement 23 to supply electrical power when required and under control of the control circuitry 25 to heat the aerosolizable material in the article (as discussed, to volatilize the aerosolizable material without causing the aerosolizable material to burn).

An advantage of locating the power source 27 laterally adjacent to the heater arrangement 23 is that a physically large power source 25 may be used without causing the device 1 as a whole to be unduly lengthy. As will be understood, in general a physically large power source 25 has a higher capacity (that is, the total electrical energy that can be supplied, often measured in Amp-hours or the like) and thus the battery life for the device 1 can be longer.

In one example, the heater arrangement 23 is generally in the form of a hollow cylindrical tube, having a hollow interior heating chamber 29 into which the article 201, 301 comprising the aerosolizable material is inserted for heating in use. Different arrangements for the heater arrangement 23 are possible. For example, the heater arrangement 23 may comprise a single heating element or may be formed of plural heating elements aligned along the longitudinal axis of the heater arrangement 23. The or each heating element may be annular or tubular, or at least part-annular or part-tubular around its circumference. In an example, the or each heating element may be a thin film heater. In another example, the or each heating element may be made of a ceramics material. Examples of suitable ceramics materials include alumina and aluminum nitride and silicon nitride ceramics, which may be laminated and sintered. Other heating arrangements are possible, including for example inductive heating, infrared heater elements, which heat by emitting infrared radiation, or resistive heating elements formed by for example a resistive electrical winding.

In one particular example, the heater arrangement 23 is supported by a stainless steel support tube and comprises a polyimide heating element. The heater arrangement 23 is dimensioned so that substantially the whole of the aerosolizable material 203, 303 of the article 201, 301 is inserted into the heater arrangement 23 when the article 201, 301 is inserted into the device 1.

The or each heating element may be arranged so that sections 103a, 103b of the aerosolizable material can be independently heated, for example in turn (over time) or together (simultaneously) as desired.

The heater arrangement 23 in this example is surrounded along at least part of its length by a thermal insulator 31. The insulator 31 helps to reduce heat passing from the heater arrangement 23 to the exterior of the device 1. This helps to keep down the power requirements for the heater arrangement 23 as it reduces heat losses generally. The insulator 31 also helps to keep the exterior of the device 1 cool during operation of the heater arrangement 23. In one example, the insulator 31 may be a double-walled sleeve which provides a low pressure region between the two walls of the sleeve. That is, the insulator 31 may be for example a “vacuum” tube, i.e. a tube that has been at least partially evacuated so as to minimize heat transfer by conduction and/or convection. Other arrangements for the insulator 31 are possible, including using heat insulating materials, including for example a suitable foam-type material, in addition to or instead of a double-walled sleeve.

The housing 9 may further comprises various internal support structures 37 for supporting all internal components, as well as the heating arrangement 23.

The device 1 further comprises a collar 33 which extends around and projects from the opening 20 into the interior of the housing 9 and a generally tubular chamber 35 which is located between the collar 33 and one end of the vacuum sleeve 31. The chamber 35 further comprises a cooling structure 35f, which in this example, comprises a plurality of cooling fins 35f spaced apart along the outer surface of the chamber 35, and each arranged circumferentially around outer surface of the chamber 35. There is an air gap 36 between the hollow chamber 35 and the article 201, 301 when it is inserted in the device 1 over at least part of the length of the hollow chamber 35. The air gap 36 is around all of the circumference of the article 201, 301 over at least part of the cooling segment 307.

The collar 33 comprises a plurality of ridges 60 arranged circumferentially around the periphery of the opening 20 and which project into the opening 20. The ridges 60 take up space within the opening 20 such that the open span of the opening 20 at the locations of the ridges 60 is less than the open span of the opening 20 at the locations without the ridges 60. The ridges 60 are configured to engage with an article 201, 301 inserted into the device to assist in securing it within the device 1. Open spaces (not shown in the Figures) defined by adjacent pairs of ridges 60 and the article 201, 301 form ventilation paths around the exterior of the article 201, 301. These ventilation paths 1 allow hot vapors that have escaped from the article 201, 301 to exit the device 1 and allow cooling air to flow into the device 1 around the article 201, 301 in the air gap 36.

In operation, the article 201, 301 is removably inserted into an insertion point 20 of the device 1, as shown in FIGS. 7 to 9. Referring particularly to FIG. 8, in one example, the aerosolizable material 203, 303, which is located towards the distal end 215, 315 of the article 201, 301, is entirely received within the heater arrangement 23 of the device 1. The proximal end 213, 313 of the article 201, 301 extends from the device 1 and acts as a mouthpiece assembly for a user.

In operation, the heater arrangement 23 will heat the consumable article 201, 301 to volatilize at least one component of the aerosolizable material from the aerosolizable material 203, 303.

The primary flow path for the heated volatilized components from the aerosolizable material 203, 303 is axially through the article 201, 301, through the chamber inside the cooling segment 207, 307, through the filter segment 209, 309, through the mouth end segment 211, 313 to the user. In one example, the temperature of the heated volatilized components that are generated from the aerosolizable material is between 60° C. and 250° C., which may be above the acceptable inhalation temperature for a user. As the heated volatilized component travels through the cooling segment 207, 307, it will cool and some volatilized components will condense on the inner surface of the cooling segment 207, 307.

In the examples of the article 301 shown in FIGS. 5 and 6, cool air will be able to enter the cooling segment 307 via the ventilation holes 317 formed in the cooling segment 307. This cool air will mix with the heated volatilized components to provide additional cooling to the heated volatilized components.

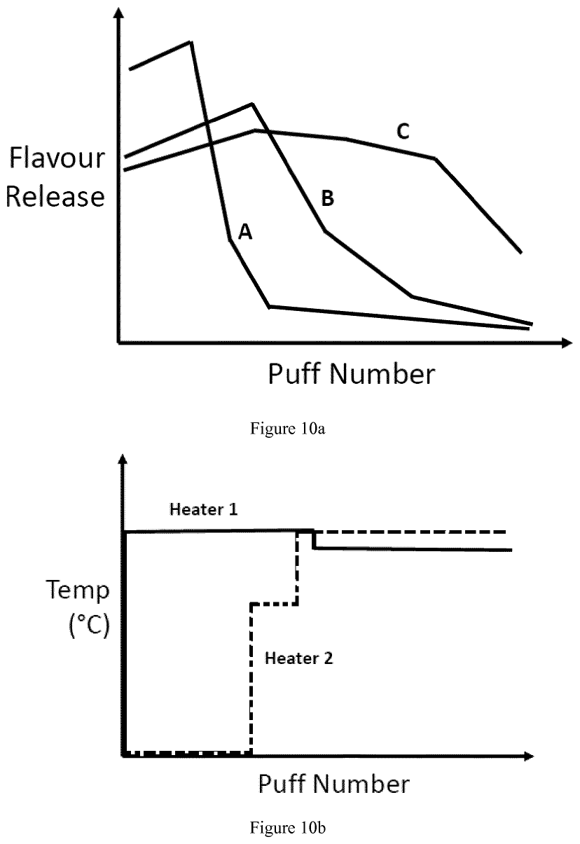

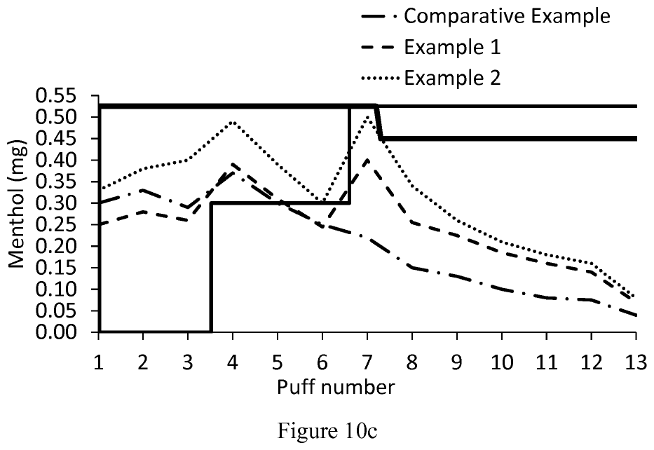

FIGS. 10a and 10b illustrate the sustained flavor delivery provided by the invention. In FIG. 10a, the flavorant delivery per puff is provided for three different aerosol generating assemblies:

In example A (comparative example), the aerosol generating article is a homogenous rod containing only un-encapsulated flavor. The whole article is heated simultaneously.

In example B (comparative example), the aerosol generating article is a homogenous rod containing only un-encapsulated flavor. However, in contrast to example A, the rod has two portions that are heated independently according to the heat profile illustrated in FIG. 10b (and exemplified in more detail in co-pending application PCT/EP2017/068804). In example C (example of the invention), the aerosol generating article comprises (i) a first portion which contains un-encapsulated flavor only and (ii) a second portion which contains un-encapsulated and encapsulated flavor. The first portion is disposed so as to be heated by “heater 1” of FIG. 10b and the second portion is disposed to as to be heated by “heater 2”.

As can be seen, the invention provides sustained flavorant delivery over a greater number of puffs.

In a further example, the aerosol generating article comprises a homogenous rod of aerosol generating material, containing a tobacco material, un-encapsulated flavorant and encapsulated flavorant. The rod has two portions that are heated independently according to the heat profile illustrated in FIG. 10b (and exemplified in more detail in co-pending application PCT/EP2017/068804).

FIG. 10c illustrates the flavorant delivery profile from two such rods (i.e. homogenous aerosol generating material, containing tobacco material, un-encapsulated flavorant and encapsulated flavorant), and a comparative rod without encapsulated flavorant. The heat profile from FIG. 10b is overlaid for ease of reference:

In the comparative example, un-encapsulated flavorant is volatilized from section 1 of the rod during the first 2 puffs and then a decrease in delivery is observed. Un-encapsulated flavorant from section 2 is released as that section is heated, with a peak delivery around puff 4. The flavorant delivery then decreases for the rest of the heating period. From consumer point of view this puff profile may result in the flavor sensory to be depleted in the initial phase of the puff profile.

In the rods that are examples of the invention (labelled Example 1 and Example 2), it can be seen that flavorant delivery is staggered and more sustained—there is greater flavorant delivery later in the consumption session. It is thought that encapsulated flavorant from the first section is released around puff 3; when compared to the comparative example, it can be seen that the flavorant delivery drop at puff 3 is ameliorated (or eliminated in the case of Example 2). It is also thought that the encapsulated flavorant in section 2 is released when that section reaches maximum temperature, resulting in the observed increase in flavorant delivery at puff 7. Consumer testing showed more sustained flavor sensory effect for the rods of Example 1 and Example 2 as compared to the Comparative Example.

In this specific example, the flavorant was menthol, and the evaluated sensorial effect was cooling.

Thus, the invention provides sustained delivery of flavorant. The invention also provides sustained sensorial effect from that flavorant. In the case where the flavorant comprises menthol, the invention provides sustained menthol delivery and sustained cooling effect.

The above examples are to be understood as illustrative examples of the invention. It is to be understood that any feature described in relation to any one example may be used alone, or in combination with other features described, and may also be used in combination with one or more features of any other of the examples, or any combination of any other of the examples. Furthermore, equivalents and modifications not described above may also be employed without departing from the scope of the invention, which is defined in the accompanying claims.

1. An aerosolizable material for use in an aerosol generating assembly, the aerosolizable material comprising a tobacco material, an un-encapsulated flavorant and an encapsulated flavorant.

2. The aerosolizable material according to claim 1, wherein the aerosolizable material is in the form of a component comprising at least two sections, and wherein the at least two sections have different compositions.

3. The aerosolizable material according to claim 2, wherein the aerosolizable material is in the form of a component comprising two sections, wherein both of the two sections comprise an un-encapsulated flavorant, and wherein only one of the two sections comprises an encapsulated flavorant.

4. The aerosolizable material according to claim 2, wherein the aerosolizable material is in the form of a component comprising two sections, wherein only one of the two sections comprises an un-encapsulated flavorant, and wherein only one of the two sections comprises an encapsulated flavorant.

5. The aerosolizable material according to claim 4, wherein the aerosolizable material is in the form of a component comprising two sections, wherein the un-encapsulated flavorant and the encapsulated flavorant are provided in different sections.

6. The aerosolizable material according to claim 4, wherein the aerosolizable material is in the form of a component comprising two sections, wherein the un-encapsulated flavorant and the encapsulated flavorant are provided in the same section.

7. The aerosolizable material according to claim 3, wherein the tobacco material is provided in either of the two sections or both of the two sections.

8. The aerosolizable material according to claim 1, wherein the encapsulated flavorant is applied to a wrapper arranged around the tobacco material.

9. The aerosolizable material according to claim 1, wherein the encapsulated flavorant provides a multi-modal flavorant release profile from the encapsulated flavorant on heating.

10. The aerosolizable material according to claim 1, wherein the aerosolizable material is in the form of a component that has a rod shape.

11. The aerosolizable material according to claim 1, wherein the un-encapsulated flavorant comprises menthol, a cooling agent, or a combination thereof.

12. The aerosolizable material according to claim 1, wherein the encapsulated flavorant comprises menthol, a cooling agent, or a combination thereof.

13. The aerosolizable material according to claim 1, wherein the encapsulated flavorant comprises an encapsulating material, and wherein the encapsulating material comprises at least one of a polysaccharide material; a cellulosic material; a gelatin; a gum; a protein material; a polyol matrix material; a gel; a wax; a polyurethane; polymerized, hydrolyzed ethylene vinyl acetate or a mixture thereof.

14. An aerosol generating article for use in an aerosol generating assembly, the article comprising:
an aerosolizable material comprising a tobacco material, an un-encapsulated flavorant, and an encapsulated flavorant; and;
a cooling element, a filter, or a combination thereof.

15. The aerosol generating article of claim 14 used in an aerosol generating assembly, wherein the aerosol generating assembly further comprising a heater arranged to heat the aerosolizable material in use to generate an aerosol.

16. The aerosol generating article used in an aerosol generating assembly according to claim 15, wherein the aerosolizable material comprises at least two sections, and wherein the assembly is configured to provide a different heat profile to each of the at least two sections of aerosolizable material.

17. An aerosol article used in an aerosol generating assembly according to claim 16, comprising at least two heaters, wherein the at least two heaters are arranged to respectively heat different sections of the aerosolizable material.

18. A method of generating an aerosol, the method comprising:
heating, in an aerosol generating assembly, an aerosolizable material, wherein the aerosolizable material comprises a tobacco material, an un-encapsulated flavorant and an encapsulated flavorant.

19. The method according to claim 18, wherein the aerosol generating material comprises at least two sections, and wherein a different heat profile is provided to each of the at least two sections of the aerosolizable material.

20. The aerosolizable material according to claim 4, wherein the tobacco material is provided in either of the two sections or both of the two sections.
